(19) United States
(12) Patent Application Publication
(10) Pub. No.: US 2007/0020713 A1
(43) Pub. Date: Jan. 25, 2007

(54) Antibodies to the fuel oxygenate mtbe and use thereof in immunoassays

(76) Inventors: Selwayan Saini, Bedfordshire (GB); Steven John Setford, Bedfordshire (GB); Sharon Louise Huntley, Bedfordshire (GB); Laurence Bourdin, Ecole (FR); Claudine Vermot-Desroches, Besancon (FR); Johnny Wudenes, Lamod (FR)

Correspondence Address: STITES & HARBISON PLLC, 1199 NORTH FAIRFAX STREET, SUITE 900, ALEXANDRIA, VA, 22314, US

(21) Appl. No.: 10/563,094

(22) PCT Filed: Jul. 1, 2004
(86) PCT No.: PCT/GB04/02807

§ 371 Date: Jun. 19, 2006

(30) Foreign Application Priority Data
Jul. 2, 2003 (GB) 0315468.9

(51) Int. Cl. G01N 33/53; C12P 21/04

(52) U.S. Cl. 435/7.92; 435/70.21; 530/388.1

(57) ABSTRACT

Immunoassay of methyl tert-butyl ether (MTBE) and related fuel oxygenates and their breakdown products is performed using a monoclonal antibody produced using a hapten with a CH₃—O—C(CH₃)₂—CH2-moiety, preferably CH₃—O—C(CH₃)₂—(CH₂)₃—CH(CH₃)—CH₂—CHO conjugated to a carrier protein.

### TECHNICAL FIELD

The present invention relates to materials, equipment and methods for determination of fuel oxygenates, e.g. members of the tert-butyl group and associated compounds. The approach, comprising an immunochemical-based (affinity-based) assay and associated reagents may be used in either the laboratory of the field for the rapid and routine diagnosis of samples suspected of containing said compounds thereby allowing rapid analytical and diagnostic information to be obtained.

Human endeavour is hugely dependent upon the consumption of fuels for power generation purposes. For example, road transport is an integral feature of today's society, essential for industry, commerce and recreational activities. Global demand for petroleum necessitates usage of large fuel storage tanks and underground oil pipelines, which present the potential for significant environmental contamination from persistent and hazardous petroleum hydrocarbons. Particularly with underground systems, leaks can remain undetected and can be transported through soils and water systems via natural dispersion mechanisms.

From the early 1970's, increasing concern about public health and environmental impacts from exhaust emissions has led to changes in the formulations of motor fuels. Revised European and US regulations have been targeted to reduce emissions from motor vehicles and improve air quality. Oxygenated compounds have been incorporated into fuels to aid combustion and reduce exhaust emissions responsible for air pollution. These provided a replacement for benzene and have aided fuel producers in reaching octane levels required by new fuel standards. The choice of oxygenate varies between countries and US states, influenced by the availability of raw materials, individual economies and political status.

Currently, the most commonly used fuel oxygenates are methyl tert-butyl ether (MTBE) and ethanol. Other oxygenates in use include ethyl tert-butyl ether (ETBE), methyl tert-amyl ether (TAME), isopropyl ether, and tert-butyl alcohol (TBA). These compounds have been primarily used to achieve mandatory fuel standards throughout the US and Europe. Whilst petroleum may comprise of >120 different compounds, leaded, unleaded and premium grade fuels may contain >15% oxygenates by volume.

MTBE has become one of the most significant environmental pollutants in recent years. Sampling results from the US Geological Survey's (USGS) National Air and Water Quality Assessment (NAWQA) Programme during 1990-1998 found high incidences of MTBE contamination in both confined and unconfined ground water aquifers and in drinking water. Approximately 60% of US drinking water is extracted from surface water systems. Whilst the potential toxicity is still debated, MTBE has an extremely low taste threshold and low-level contamination has led to losses of drinking water supplies. 71% of the water supply of Santa Monica, Calif., has been tainted with MTBE, requiring the importation of water supplies from outside of the city limits at an annual cost of $3.5 million per annum. MTBE has been increasingly identified as the primary threat to European ground water reservoirs, which supply 60-70% of all European drinking water.

The main source of environmental oxygenate contamination is from oxygenate-blended petroleum, through leaking underground storage tanks (LUSTs), transfer spillage, petroleum facilities and/or accidental spills. Poor management of stored fuel and subsequent leakages have resulted in major, global environmental contamination from petroleum and related components, typically BTEX compounds (benzene, toluene, ethylbenzene and m-, o-, p-xylenes), low weight alkanes, such as n-pentane, n-hexane, and fuel oxygenates. MTBE has particular environmental significance due to its high solubility in water, with numerous incidences of ground water and/or drinking water contamination. Surface water systems may become contaminated through ground water, atmospheric deposition, storm water run-off and direct releases by industrial and recreational activities.

### BACKGROUND ART

Traditionally, environmental analyses of petroleum and related compounds are performed using laboratory-based methods such as gas or liquid chromatography allied to suitable detection methodologies. The two most widely used analytical methods for detection of fuel oxygenates are EPA Method #8260 (Volatile organic compounds by Gas Chromatography/Mass Spectroscopy (GS/MS) and Method #8015 (Non-halogenated organics using Gas Chromatography/Flame Ionisation Detector (GC/FID). Additionally, there are problems with sample collection methods and sample preservation for these laboratory-based techniques. Whilst a wide range of analytical techniques are available, these remain primarily laboratory-based. Due to escalating reports of petroleum and specifically MTBE contamination across the US and Europe, there remains an urgent need for simple and accurate diagnostic tools for in-situ analysis and continuous monitoring applications.

Immunoassays

The specific, sensitive and low-cost decentralised determination of a number of different types of common environmental contaminant has been achieved through the use of affinity-based assays, notably immunoassays. Immunoassays allow the detection and measurement of target compounds using specific binding characteristics of antibodies. They can be found in a wide variety of formats and are increasingly being developed and employed for environmental monitoring purposes. These have acquired wide acceptance in the USA, with the US EPA recognising and releasing official ELISA methods for the determination of certain compounds, e.g. certain pesticides, PAHs and PCBs. ‘Rapid’ immunoassay test kits are commercially available for a wide range of analytes, which provide a relatively inexpensive, rapid (<2 h), sensitive screening method for analyte detection, commonly in aqueous and soil matrices. Castillo et al. (Castillo, M. et al. (1998). Environmental Science and Technology, Vol. 32914), pp. 2180-2184) have evaluated the immunoassay test kits for the accurate and sensitive determination of pentachlorophenol, carcinogenic PAHs and BTEX compounds within industrial effluents.

The fundamental principle of immunoassays (IAs) is that they utilise biologically generated immunoglobulin proteins—antibodies (Ab)—which react with specific target compounds—namely the target analyte, commonly referred to as the ‘antigen’ (Ag) to form antibody-antigen complexes (Ab-Ag). All IAs are based on the selectivity and sensitivity of this Ab-Ag reaction. Due to the wide range of variations in assay design, there is no universal, consistent classification system, or terminology. Each immunoassay will need to be specifically developed to the target of interest, i.e. the antigen. The Ab is the key reagent and these generally determine the assay's characteristics. Ab-Ag binding arises from structural complementarity between the two molecules, stabilised by binding through a combination of Van der Waals forces, electrostatic interactions, hydrogen bonding and hydrophobic interactions.

Antibody Production

The initial stimulation of Immunoglobulin G (IgG) antibodies is achieved through injecting animals, commonly mice, rabbits or sheep, with the target immunogen (antigen). The in vivo administration of an immunogen stimulates B-lymphocyte cells to produce and secrete antibodies into the blood stream that are capable of binding to (and, with the help of other factors in the immune response, destroying) the invading blood-borne foreign body. Since each stimulated B-lymphocyte cell will produce a unique antibody ‘clone’ exhibiting a specific binding reaction, and hence affinity, to the target analyte, a ‘polyclonal’ mixture of antibodies derived from all of the individual Ab secreting cells, is elaborated within the serum. This polyclonal antiserum can be utilised at this stage, although the IgG fraction will contain many antibodies of differing specificities, many of which may be irrelevant for IA purposes (Edwards, R. (1996) (Ed.) Immunoassays. Essential data Series. John Wiley and Sons, Chichester, UK). A purification step is generally necessary to increase performance and limit the possibility of activity from those irrelevant fractions.

With polyclonal Ab preparations, there are significant batch-to-batch variations in the quality of the antiserum and the source of the preparation will cease on death of the animal host. This is a particular problem in immunoassay manufacture when product consistency is paramount for maintaining a valid analytical/diagnostic tool. This issue has been overcome through the development of monoclonal antibody production methods, which exploit the use of neoplastic multiple myeloma ‘tumour’ cells—essentially Ab secreting cells that undergo uncontrolled and rapid cell division. Individual Ab secreting cells are isolated from the animal host and fused with these myeloma cells, to form hybridoma cells. These, with careful cultivation, act as effectively immortal cell lines for the production of individual cloned (monoclonal) Ab preparations. Generally, vast numbers of hybridoma cells are generated and then screened for Ab-Ag binding efficacy. The high purity, homogeneity and cloning ability of MAbs enables easier purification and subsequent labelling of these highly specific antibodies. Although costs and practical investments are initially high, these are now the preferred and established practice for IA design.

A significant issue arises in that most organic pollutants are of insufficient molecular weight to engender an immunogenic response. Molecules with a molecular weight of <3000 are not immunogenic and those <5000 may be too weak to induce an adequate immunogenic response. In these cases, the molecules must be conjugated to a much larger carrier protein in order to provoke an immune response. Only those antibodies binding specifically to the haptenic determinant, as opposed to the carrier protein will be of diagnostic use. Theoretically, various immunogenic carriers can be used, the most common being bovine serum albumin (BSA), human serum albumin (HSA), rabbit thyroglobin and keyhole limpet haemacyanin (KLH). In fact, this approach is difficult, particularly with small haptens. A very extensive screening program may be required, without guarantee of success.

Immunoassay Design

There are many possible immunoassay formats reported in the literature that exploit the fundamental principle of quantifying the extent of Ab-Ag binding. The method most used for field-based determinative purposes is termed the indirect competitive assay format. The method is indirect in that one of the immunoreagents must be labelled in order to visualise the extent of Ab-Ag binding and hence quantify the amount of target analyte in the sample solution. The choice of label remains largely dependent on the specific characteristics and expected concentrations of the target analyte.

Suitable labels include enzymes, fluorochromes and radioisotopes, the latter being less widely used in recent years. Enzymes can also be selected to convert non-fluorescent substrates to fluorescent products, e.g. alkaline phosphatase. Photoluminescent compounds in immunoassays (FIAs) can provide even greater sensitivity that calorimetric substrates. Fluoroscein isothiocyanate (FITC) is usually the label of choice for immunofluorescent IAs, as coupling procedures are straightforward works with almost all antibodies. Additional labels commonly used include tetramethylrhodamine isothiocyanate (TRITC), Texas Red (TR) or phycoerythrin (PE).

Enzyme Linked Immunoassays (ELISA)

Enzyme linked immunosorbent assays (ELISAs) are based on the combination of selective antibodies with sensitive enzymes that react with a substrate to produce a detectable colour change, e.g. commonly horseradish peroxidase (HRP), or alkaline phosphatase. Specific enzymes are incorporated to link to target contaminants and through enzymatic actions on the colouring agent (chromogen), enabling both qualitative and quantitative analysis through the catalytic capability of the enzymes. Many regulated contaminants, such as pesticides, polyaromatic hydrocarbons (PAHs) and other organic pollutants can now be detected on-site using available test kits utilising enzyme linked immunoassay technology. A number of solid supports can be used as a means of separation in an ELISA system. Solid support within ELISA systems can be the traditional microtitre plate systems, coated tubes or using covalently bound antibodies to magnetic or latex particles. Due to their relative simplicity, flexibility, speed and cost, IAs are now generally considered an effective and suitable form of analysis, suitable for both laboratory and field diagnostics. Such IA systems are capable of detecting very low levels of contamination, in some cases as low as the ng/L or ppb level (see for example the website of Strategic Diagnostics Incorporated, http://www.sdix.com.

For petroleum contaminants, a number of IA based field kits are commercially available for on-site analysis, e.g. the Strategic Diagnostics Incorporated RaPID Assay Petroleum Fuel Kits. These field kits utilise Abs bound onto microscopic particles, for which results are interpreted using a portable microprocessor controlled spectrophotometer or photometer. Detection levels can be at μg/L (ppm) or ng/L (ppb) concentrations, depending on the analyte in question and product used. Reportedly, results can be obtained in less than two hours. However, whilst the IA aspects can be accomplished in some cases in <60 minutes, extraction procedures are necessary for soil analysis, which increase the complexity and time scale of analysis.

There is extreme current interest in MTBE and related fuel oxygenates—ETBE, TAME, TBA etc.—hereafter referred to as the tert-butyl oxygenate family, and their environmental impact. Despite this fact there is no prior art concerning the development of immunochemical methods for determination of such compounds. It is worthy of note that commercially available test kits to the other major classes of petroleum species, such as the BTEX compounds, TPHs (total petroleum hydrocarbons) and PAHs (polynuclear aromatic hydrocarbons) do currently exist.

### DISCLOSURE OF INVENTION

This invention therefore pertains to the processes, technologies and associated knowledge associated with the generation of immunoreagents and the development of immunoassays for the antibody-based determination of members of the tert-butyl oxygenate family. The benefits of such an approach lie in the simplicity of the assay procedure and amenability of said procedure to decentralised usage. The method offers the following benefits over current MTBE determination methods:
The simplicity of the procedure and requirement for simple instrumentation (optical reader, electrochemical monitoring device or such other signal interrogation device) renders the method amenable to decentralised operation.
High sample throughput: achieved through the ability to assay a large number of samples simultaneously.
A high degree of assay specificity and sensitivity due to the binding complementarity and binding affinity between the antibody and target analyte(s).
Assay rapidity, typically in the region of 1-2 h.
In situ sample determination removes the requirement for transportation of samples to a centralised facility and the concomitant degradation of sample during transportation and storage.
Despite the costs associated with antibody preparation, particularly monoclonal antibody preparation, the small amounts of Ab material and other reagents required for each assay measurement results in low assay costs relative to the costs associated with running complex instrumentation at dedicated centralised analytical facilities.
Operator costs are reduced due to the speed/throughput of the assay and the lower levels of training required relative to their laboratory counterpart.
   In order to generate antibodies with binding specificities directed towards the tert-butyl oxygenate family, account must be made of the low molecular weight of these compounds. Research in our laboratories has focused on producing tert-butyl oxygenate-protein conjugates capable of engendering an immunological response in a host animal species.

One critical aspect of this invention relates to the synthesis and utilisation of tert-butyl oxygenate analogues for immunogen production. These analogues may be polymers (dimers, trimers, polymers etc.), produced by polymerisation of two or more tert-butyl oxygenate monomers, to form extended repeated tert-butyl oxygenate polymer chains, or alternatively, individual molecules exhibiting structural and functional characteristics of the tert-butyl ether target compound in association with a second entity that acts as a spacer between the tert-butyl ether functionality and associated carrier compound. Additionally, these compounds will also contain a functionality located away from the tert-butyl ether moiety that can be used for carrier compound conjugation purposes, such as conjugation to suitable carrier proteins. Such entities, henceforth referred to as [tert-butyl oxygenate]ₙ-carrier protein conjugates (i.e. tert-butyl ether polymer structures and tert-butyl ether-spacer compounds) were found to elicit the desired immunological response in host animals, with recovery of antibody with specificity and high binding affinity for both the tert-butyl oxygenate polymer and original monomer compound. Simple conjugation of members of the tert-butyl oxygenate family to standard carrier proteins yielded antibodies with significantly weaker antigen binding affinity.

In one preferred embodiment the invention provides a method of generating antibodies useful for assaying a sample for fuel oxygenates comprising (i) conjugating a hapten having a CH₃—O—C(CH₃)₂—CH₂-moiety to a carrier protein to produce a conjugate; (ii) injecting the conjugate into an animal; (iii) harvesting antibody-synthesising cells from the animal; (iv) fusing the antibody-synthesising cells with myeloma cells to form hybridoma cells; (v) cultivating the hybridoma cells; (vi) screening the cultivated cells to find desired cells producing monoclonal antibodies capable of binding methyl tert-butyl ether (“MTBE”); and (vii) cultivating said desired cells and harvesting said monoclonal antibodies.

This invention further relates to the use of methodologies whereby the specific binding capabilities of these antibodies, produced using either the tert-butyl oxygenate polymer-carrier protein or tert-butyl oxygenate monomer-carrier protein immunogen route, are exploited for the creation of immunodiagnostic methods for the determination of members of the tert-butyl oxygenate family. The immunoassay format may vary in nature but relates to the use of immunochemical assay formats and specific assay labels that are able to visualise the extent of Ab-Ag binding and hence lead to the determination of the tert-butyl oxygenate family members.

The assay format may be competitive or non-competitive in operation and may include alternative embodiments of either approach, such as the use of particulates, such as latex beads, magnetic beads and the well described lateral flow assay format. The assay label may be linked directly to one or more of the immunoassay reagents, such as antibody or antigen analogue, or may be introduced by alternative means, such as via the well-known streptavidin-biotin binding complex, or through conjugation of the label to a second antibody or binding component with binding specificity directed towards structures on the primary anti-tert-butyl oxygenate antibody. The label may be, but is not limited to an enzyme, chromophore, fluorophore or other optically detectable agent, chemically active agent, electrochemically active agent or other such suitable compound in which the specific properties of the label can be used to visualise the Ab-Ag binding process. In the case of enzyme labels, the consumption of active substrate and/or the generation of active product or other enzyme mediated effect may be used to produce the assay response. The transduction process, which will be dependent upon the selected assay label, may be selected from optical, electrochemical and other appropriate methods. Integration of the immunoassay with appropriate transduction methodologies to produce dedicated sensing tools is a further clear embodiment of the invention described herein. The present invention may also relate to any assay in which affinity is used as the recognition method, such as use of synthetically produced ligands (‘molecularly imprinted polymers’) or any other ligand with the required binding specificity.

Whilst it is evident that the invention is suitable for decentralised determination of members of the tert-butyl oxygenate family using the indirect immunochemical assay format, it is evident that the methodologies herein described may be equally applied to direct immunochemical assay methods. Direct methods are able to visualise the Ab-Ag binding process without the aid of associated assay labels by use of appropriate transduction methodologies. Such methodologies would include, but would not be limited to: surface plasmon resonance (SPR) devices, evanescent wave devices, quartz crystalline microbalance (QCM) devices, surface/bulk acoustic wave devices, field-effect transistors or any other such methodology that may be considered direct in operation.

### BRIEF DESCRIPTION OF DRAWINGS

FIG. 1 is a graph of antibody concentration vs antigen concentration for a checkerboard assay for determining the optimal coating Ag and primary Ab concentrations.

FIG. 2 shows a standard ELISA result comparison with spiked MTBE samples in PBS.

FIG. 3 resembles FIG. 2 and shows results for repetitions on different days, using fresh solutions of all reagents.

FIG. 4 is a graph showing the cross-reactivity of the antibody to a range of compounds.

FIG. 5 and FIG. 6 are bar charts showing results of MTBE immunoassay using magnetic beads, with MTBE concentrations ranging from 0-5000 mg/l (FIG. 5) and 0-0.005 mg/l (FIG. 6)

### MODES FOR CARRYING OUT THE INVENTION

Some specific embodiments of the invention will now be described in detail by way of example.

### Example 1

An indirect competitive immunoassay with specificity towards MTBE has been constructed and tested with representative samples. The assay is centred on the competition between microtitre well wall immobilised MTBE-spacer-BSA polymer (7-methoxy-3,7-dimethyloctanal-BSA—the ‘coating antigen’), free MTBE in the sample and free anti-MTBE/MTBE-spacer antibody.

Anti-MTBE/MTBE-spacer antibody was first generated by conjugating 7-methoxy-3,7-dimethyloctanal to BSA carrier protein. The resultant conjugate was injected into a mouse host and antibody synthesising cells harvested at the appropriate time. The antibody synthesising cells were fused with myeloma cells to form hybridoma cells which were cultivated and the antibody specificity of each hybridoma assessed with respect to binding to MTBE and 7-methoxy-3,7-dimethyloctanal. Those hybridomas expressing antibody of the required specificity were propagated and used as a source of anti-MTBE/7-methoxy-3,7-dimethyloctanal antibody for immunoassay applications.

A competitive immunoassay is described in this particular embodiment. Free MTBE antigen present in the sample was allowed to compete with the immobilised coating antigen for anti-MTBE/7-methoxy-3,7-dimethyloctanal antibody. The quantitative nature of the assay is evident from the fact that the final signal response generated by the assay is dependent upon free MTBE-Ab binding. In essence, the lower the concentration of free MTBE in the sample solution, the greater the extent of antibody binding to the coating antigen. On washing the microtitre wells, free MTBE-antibody complexes are displaced from the well. An antibody-specific enzyme tracer is then added to the system. Following the binding and washing steps, residual enzyme activity, which is inversely proportional to the original free MTBE concentration, is then determined by adding enzyme substrate and recording the optical density of the coloured enzymic product after a suitable period of colour development. Low concentrations of MTBE in the sample gave an inversely proportional large signal output, whilst high concentrations of free MTBE in solution resulted in a reduced assay signal.

Microtitre Well Preparation

Wells were prepared by adding 100 μl of coating antigen (500 ng/ml) and then incubated at 4° C. for 24 hours. Plates were then aspirated and washed with wash solution (0.01% v/v Tween 20 in Reverse Osmosis (RO) water) at 300 μl/well. After each wash step, plates were inverted and blotted against clean paper towelling. Unless otherwise stated, plates were blocked using 5% v/v BSA blocker buffer and incubated at room temperature (RT) for 2 hours. Plates were then aspirated and two wash steps performed. Dried plates were then sealed and stored with desiccant at 4-8° C. prior to use.

ELISA Immunoassay Procedure

A 100 μl volume of MTBE-containing sample was added to each microplate well followed by 10011 of biotinylated anti-MTBE/7-methoxy-3,7-dimethyloctanal Ab. The plates were then sealed and incubated for 1 hour at RT. The aspiration and wash step, as described for plate preparation, was repeated ×4. Streptavidin-HRP (100 μl per well) was added, the plates sealed and incubated at RT for a further 30 minutes. Aspiration and 4 further wash steps were made. To each well, 100 μl of the HRP enzyme substrate TMB (3,3′,5,5′ tetramethyl benzidine) was added. The plates were then covered and incubated for 10 minutes. At the end of the colour development time, the colour reaction was stopped by the addition of 100 μl 2M H₂SO₄ and the OD₄₅₀ immediately measured. Any necessary dilutions were made with RO water.

Results

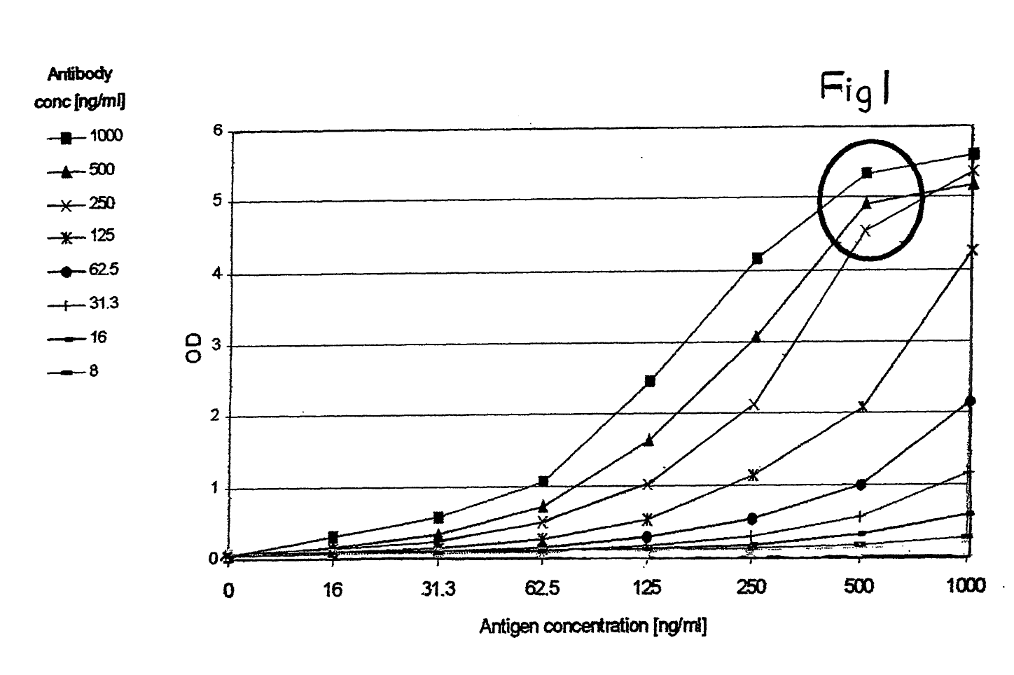

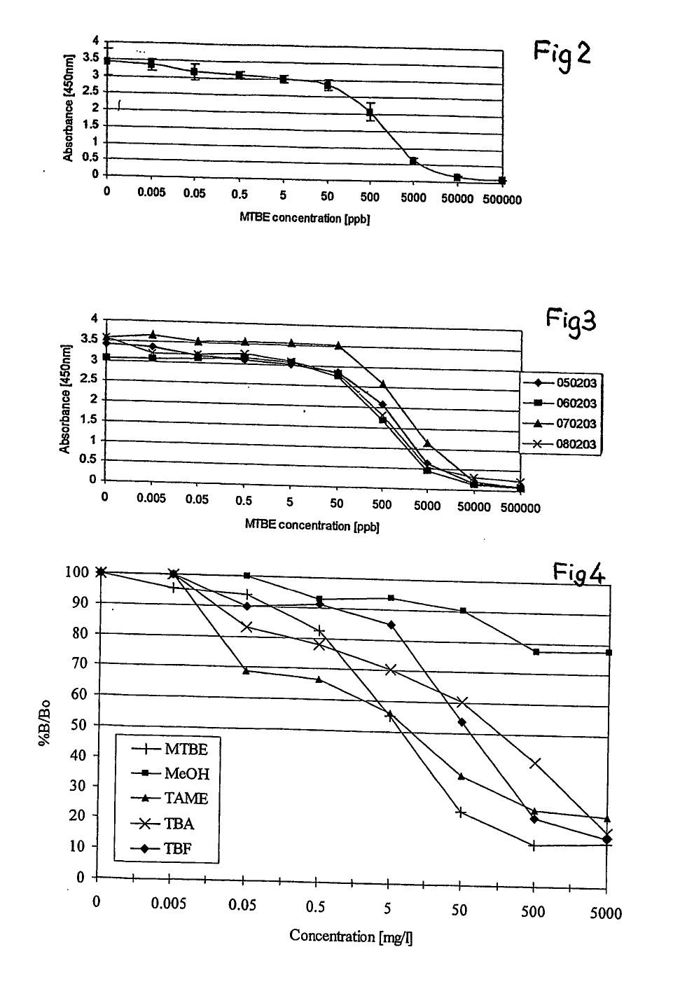

Optimal concentrations of both Ag and Ab (biotinylated and native) were determined by checkerboard assay (FIG. 1). The optimal concentrations of coating antigen conjugate and biotinylated Ab were all found to be 500 ng/ml. As shown in the figure, the highest signal was achieved using 500 ng/ml coating antigen and 500 □g/ml anti-MTBE/7-methoxy-3,7-dimethyloctanal antibody. A 500 ppm MTBE stock solution was prepared in 100 mM phosphate buffered saline (PBS, pH 7.4) and serially diluted (1/10) in 100 mM PBS to a final concentration of 0.5 ppb. A competitive ELISA was then performed. FIG. 2 shows a reproducible sigmoidal curve of assay response (OD₄₅₀) as a function of increasing free MTBE concentration. As the ELISA is a competitive assay, the OD₄₅₀ is inversely proportional to increasing concentrations of free MTBE. The dynamic range of the assay was found to be 50-5000 ppb for free MTBE. The assay was repeated on different days and with fresh solutions of all reagents. Results are shown in FIG. 3.

The dynamic range of the assay was found to be 50-5000 ppb for free MTBE. Plates 050203 and 060203 were blocked with 5% v/v BSA blocking buffer. Plates 070203 and 080203 were blocked with 1% BSA blocking buffer.

### Example 2

### Anti-MTBE Monoclonal Antibody Cross-Reactivity

The cross-reactivity of the anti-MTBE antibody prepared in Example 1 towards a range of tert-butyl related compounds was tested. The results are shown in FIG. 4. An indication of the binding affinity between the antibody and the various test compounds can be obtained by determining the concentration of analyte (IC₅₀) required to inhibit the maximum assay signal (B₀) by 50% (i.e. B/B₀=50%). The lower the concentration value, the greater the binding affinity, since a lesser amount of analyte is required to depress the assay signal by one half. IC₅₀ values of ˜10 mg/l were recorded for MTBE and TAME, whilst IC₅₀ values of ˜60 mg/l and ˜180 mg/l were recorded for TBF (tert-butyl formate) and TBA respectively. The antibody showed no significant cross-reactivity to the methanol control. This data suggests that immunoassays can be constructed, using antibody preparations prepared according to the methods disclosed in this document, to identify MTBE and also compounds other than MTBE in test samples. This may prove advantageous in many different situations, such as the case where a previous MTBE contamination may have occurred and in which MTBE breakdown products are present.

### Example 3

### Magnetic Bead Format

A stock solution of commercially available tosylactivated paramagnetic beads (Hydrophobic Dynabeads M280 with p-toluene-sulfonyl [tosyl] groups attached, Dynal Biotech, Bomborough, Wirral, UK; bead concentration of 2×10⁹ beads/ml, equivalent to approximately 30 mg beads/ml) was obtained. Beads were washed and then diluted in 0.1M borate buffer pH 9.5, according to manufacturer's instructions, to yield a solution containing 1×10⁷ beads. A 3 μg quantity of coating antigen was added to the preparation to yield a solution at pH 9.5, containing 3 μg coating antigen and 1×10⁷ beads in 1 ml of incubation solution. The beads were then incubated for 24 h at 37° C. to bind the coating antigen to the beads. Beads were then washed (×2) in 0.1M phosphate buffer (containing 0.1% w/v BSA) and ×2 in 0.1M Tris (also in 0.1% w/v BSA) according to manufacturer's instructions and made up to a final volume of 1 ml in 0.1M phosphate buffer, pH 7.4.

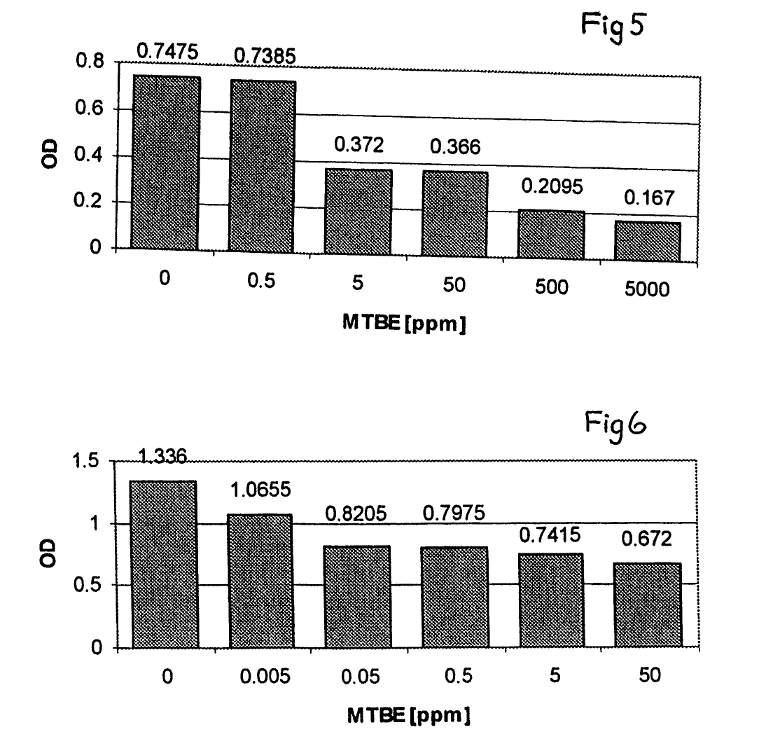

Immunoassays were then conducted in BSA-blocked microtitre plates. A 100 μl volume of the coated magnetic bead preparation was added to the wells, followed by 50 μl volumes of primary antibody (varying concentrations) and free MTBE (0-5000 mg/l). Following a 25 min. incubation step, the beads were washed as per the previously described procedure to remove residual unbound reagents. Enzyme labelled conjugate was then added to the bead preparation and a further 25 min. incubation performed. Beads were again washed as per the previously described procedure and 500 μl TMB HRP enzyme substrate added to each well. Colour development was stopped after 10 min. by addition of 100 μl 2M H₂SO₄ and the OD₄₅₀ immediately measured. Results are shown below in FIGS. 5 and 6. The quantitative nature of the assay is immediately apparent on observing the magnitude of the OD₄₅₀ readings as a function of free MTBE concentration. The assay is able to yield quantitative information across the MTBE range 0-5000 mg/l and is able to distinguish between samples containing 0 and 0.005 mg/l MTBE.

1. A method of generating antibodies useful for assaying a sample for fuel oxygenates comprising (i) conjugating a hapten having a CH3O—C(CH3)2-CH2-moiety to a carrier protein to produce a conjugate; (ii) injecting the conjugate into an animal; (iii) harvesting antibody-synthesising cells from the animal; (iv) fusing the antibody-synthesising cells with myeloma cells to form hybridoma cells; (v) cultivating the hybridoma cells; (vi) screening the cultivated cells to find desired cells producing monoclonal antibodies capable of binding methyl tert-butyl ether (“MTBE”); and (vii) cultivating said desired cells and harvesting said monoclonal antibodies.

2. A method according to claim 1 wherein said hapten is: CH3O—C(CH3)2-CH2-X—B
where X is a spacer and B is a group capable of binding to a carrier protein.

3. A method according to claim 2 wherein the spacer X comprises a hydrocarbon chain of 2-8 carbon atoms.

4. A method according to claim 3 wherein the spacer X is: —CH2.CH2.CH(CH3).CH2-

5. A method according to claim 2, wherein the binding group B is —CHO.

6. A method according to claim 1 wherein the carrier protein is selected from bovine serum albumin, human serum albumin, rabbit thyroglobin and keyhole limpet haemacyanin.

7. A method according to claim 1 wherein the monoclonal antibodies exhibit binding to methyl tert-butyl ether, ethyl tert-butyl ether, methyl tert-amyl ether and tert-butyl alcohol.

8. A monoclonal antibody capable of binding methyl tert-butyl ether as produced by the method of claim 1.

9. A method of assaying a sample for fuel oxygenates and their breakdown products comprising generating antibodies by a method according to claim 1 and carrying out an immunoassay using said antibodies.
